(19) United States
(12) Patent Application Publication
(10) Pub. No.: US 2024/0043615 A1
(43) Pub. Date: Feb. 8, 2024

(54) AMINO ACID POLYMER-PLATINUM ANTICANCER DRUG CONJUGATES

(71) Applicant: THE METHODIST HOSPITAL SYSTEM, Houston, TX (US)

(72) Inventors: Haifa SHEN, Bellaire, TX (US); Dongfang YU, Pearland, TX (US); Yongbin LIU, Houston, TX (US)

(21) Appl. No.: 18/265,331

(22) PCT Filed: Dec. 3, 2021
(86) PCT No.: PCT/US2021/061769

§ 371 Date: Jun. 5, 2023

Related U.S. Application Data
(60) Provisional application No. 63/121,466, filed on Dec. 4, 2020.

(51) Int. Cl. C08G 69/10; A61K 45/06; A61P 35/00

(52) U.S. Cl. CPC C08G 69/10; A61K 45/06; A61P 35/00

(57) ABSTRACT

The present disclosure describes compositions for the treatment of oncological disorder, more particularly to amino acid polymer conjugates with platinum agents for the treatment of cancers.

### CROSS-REFERENCE TO RELATED APPLICATIONS

This application claims the benefit of priority to U.S. Provisional Application No. 63/121,466, filed Dec. 4, 2020, the disclosure of which is incorporated herein by reference in its entirety.

### BACKGROUND

Platinum-based agents, which are coordination complexes of platinum, are among some of the most commonly used chemotherapy drugs for cancer treatment, with these drugs being used to treat almost half of people receiving chemotherapy for cancer. In this form of chemotherapy, popular drugs include cisplatin, oxaliplatin, and carboplatin, but several have been proposed or are under further development. However, these drugs tend to cause severe toxicity to the body, limiting the overall drug dosage that may be administered. In total, these drugs can cause a combination of more than 40 specific side effects which can include neurotoxicity, which is manifested by peripheral neuropathies including polyneuropathy. As a result of lower drug administration, most patients develop resistance to platinum based drugs during treatment. There is a clear need for the development of platinum-based anticancer agents with higher cancer cell killing activity and lower system toxicity. The present disclosure addresses these needs along with other needs.

### SUMMARY

In accordance with the purposes of the disclosed materials and methods, as embodied and broadly described herein, the disclosed subject matter, in one aspect, relates to compounds, compositions, and methods of making and using compounds and compositions.

Thus, in one aspect, the present disclosure provides an amino acid polymer comprising:
a plurality of glutamic acid residues, salts thereof, or combinations thereof;
a plurality of aspartic acid residues, salts thereof, or combinations thereof; and
a plurality of residues having the structure

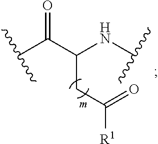

wherein:
m is 1 or 2;
R¹ is selected from

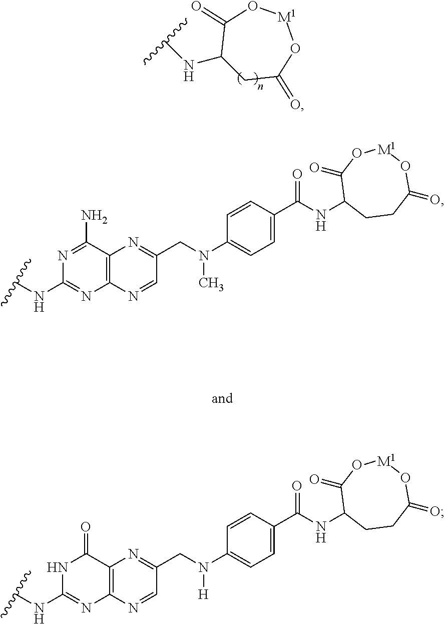

n is 1 or 2;
M¹ is Pt(NH₂R²)(NH₂R³);
R² and R³ are independently hydrogen, alkyl, or aryl; or
R² and R³ are brought together to form a cycloalkyl or heterocycle ring which may be optionally substituted with 1 or 2 alkyl groups.

In another aspect, the present disclosure provides an amino acid polymer comprising:
a plurality of glutamic acid residues, salts thereof, or combinations thereof; and
a plurality of aspartic acid residues, salts thereof, or combinations thereof;
wherein the amino acid polymer is conjugated to one or more M 2 groups having the structure:

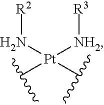

wherein each of the wavy lines represents a point of covalent attachment to the amino acid polymer, and wherein all other variables are as defined herein.

Also provided are pharmaceutical compositions comprising an amino acid polymer as described herein and a pharmaceutically acceptable carrier.

In another aspect, a method of treating a cancer in a subject in need thereof is provided comprising administering a therapeutically effective amount of an amino acid polymer or a pharmaceutical composition as described herein. In some embodiments, the amino acid polymer or pharmaceutical composition is administered in combination or alternation with one or more additional anticancer agents.

Also provided is a method of treating a cancer in a subject in need thereof, wherein the subject has been previously administered a platinum anticancer agent, the method comprising administering a therapeutically amount of an amino acid polymer or a pharmaceutical composition as described herein. In some embodiments, the cancer has developed resistance to the platinum anticancer agent. In some embodiments, the platinum anticancer agent is selected from cisplatin, carboplatin, oxaliplatin, or nedaplatin.

In another aspect, a method of treating a cancer in subject in need thereof is provided, wherein the cancer has developed resistance to a platinum anticancer agent, the method comprising administering a therapeutically effective amount of an amino acid polymer or a pharmaceutical composition as described herein. In some embodiments, the platinum anticancer agent is selected from cisplatin, carboplatin, oxaliplatin, or nedaplatin.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF DRAWINGS

FIG. 1 provides UV-visible spectra of P(G-A), Asp-Pt, and P(G-A)-Asp-Pt in water.

FIGS. 2A and 2B provide a ¹H-NMR spectrum (FIG. 2A) and ¹³C-NMR spectrum (FIG. 2B) of P(G-A)-Asp-Pt.

FIGS. 3A and 3B show mass spectrograms of P(G-A)-Asp-Pt in positive-ion (FIG. 3A) and negative-ion (FIG. 3B) mode.

FIGS. 4A and 4B show the size and zeta potential of P(G-A)-Asp-Pt. FIG. 4A shows the transmission electron microscopy (TEM) image of P(G-A)-Asp-Pt. The scale bar indicated 100 nm. FIG. 4B shows the size distribution by intensity of P(G-A)-Asp-Pt. FIG. 4C shows the zeta potential measurement of P(G-A)-Asp-Pt.

FIGS. 5A and 5B show the cytotoxicity of oxaliplatin and P(G-A)-Asp-Pt in mouse colorectal cancer CT26 cells. FIG. 5A shows the cell viability of CT26 cells with respect to incubation time in minutes that were administered 25 μg Pt/mL of oxaliplatin or P(G-A)-Asp-Pt. FIG. 5B shows the half-maximal inhibitory concentration (IC₅₀) of oxaliplatin or P(G-A)-Asp-Pt in CT26 cells in different incubation times.

FIGS. 6A, 6B, and 6C show the cytotoxicity of oxaliplatin or P(G-A)-Asp-Pt in different cancer cell lines. The cell viability of CT26 cells (FIG. 6A), A2780 human ovarian cancer cells (FIG. 6B), and MDA-MB-231 human breast cancer cells (FIG. 6C) was analyzed. Each cancer cell line was incubated with varying platinum concentration (μg/mL) for oxaliplatin and P(G-A)-Asp-Pt for 30 minutes.

FIGS. 7A and 7B show the cytotoxicity of different platinum derivatives in CT26 cells. FIG. 7A shows the cell viability of CT26 after incubating with varying platinum concentration (μg/mL) for oxaliplatin, P(G-A)-Asp-Pt, P(G-A)-Pt, PAA-Asp-Pt, PGA-Asp-Pt, and Asp-Pt for 30 minutes. FIG. 7B shows the cell viability of CT26 after incubating with varying platinum concentration (μg/mL) for P(G-A)-Asp-Pt, P(G-A)-Glu-Pt and P(G-A)-D-Asp-Pt for 30 minutes.

FIGS. 8A and 8B show the cytotoxicity of oxaliplatin and P(G-A)-Asp-Pt in different drug resistance cancer cell lines. Cell viability of cisplatin resistant A2780 cells (A2780-Cis, FIG. 8A) and multiple drug resistant MDA-MB-231 cells (MDA-MB-231/MDR, FIG. 8B) cells after treatment with cisplatin, oxaliplatin or P(G-A)-Asp-Pt at different concentration of Pt for 30 minutes.

FIG. 9 shows cytotoxicity of P(G-A)-Asp-Pt in human non-small cell lung cancer A549 cells and normal human bronchial epithelial HBEC3-KT cells. A549 and HBEC-KT cells were incubated with P(G-A)-Asp-Pt at 20 μg (Pt)/mL for different times.

FIGS. 10A and 10B show the cellular update of P(G-A)-Asp-Pt. FIG. 10A shows the viability of CT26 cells pre-treated with inhibitors for endocytosis/micropinocytosis (dynasore, chlorpromazine, genistein and cytochalasin D) for 40 min before incubation with P(G-A)-Asp-Pt at 25 μg (Pt)/mL for 30 min. FIG. 10B shows the Pt concentration in CT26 cells after treatment with oxaliplatin or P(G-A)-Asp-Pt after incubation with oxaliplatin or P(G-A)-Asp-Pt at 10 μg (Pt)/mL for 1 h.

FIGS. 11A and 11B show lysosomal changes after treatment with P(G-A)-Asp-Pt. FIG. 11A shows the Mean Fluorescent Intensity (MFI) of Lysotracker signal in CT26 cells after incubation with P(G-A)-Asp-Pt. FIG. 11B shows the quantitation of MFI from FIG. 11A.

FIGS. 12A-12D show the cancer cell necrosis induced by P(G-A)-Asp-Pt. The percentage of apoptosis (FIG. 12A) and necrosis (FIG. 12B) cells of CT26 cells after treatment with oxaliplatin or P(G-A)-Asp-Pt at 15 μg Pt/mL at different times. FIG. 12C shows the time-dependent morphological changes in CT-26 cells after incubation with P(G-A)-Asp-Pt.

FIGS. 13A and 13B show the plasma pharmacokinetic of oxaliplatin and P(G-A)-Asp-Pt in mice. FIG. 13A shows the Pt concentration in plasma versus time after injection of oxaliplatin or P(G-A)-Asp-Pt. FIG. 13B shows the area under the curve (AUC) of FIG. 13A in different time points.

FIGS. 14A and 14B show the pharmacokinetics of oxaliplatin and P(G-A)-Asp-Pt in tumor tissues. FIG. 14A shows the Pt concentration in tumors versus time after injection of oxaliplatin or P(G-A)-Asp-Pt. FIG. 14B shows the AUC of FIG. 14A in different time points.

FIGS. 15A-15C show the anti-tumor efficacy of oxaliplatin or P(G-A)-Asp-Pt in different types of cancer. Tumor growth curves of CT26 tumor (FIG. 15A), A2780 tumor (FIG. 15B) and A2780 cisplatin-resistant tumor (FIG. 15C) in mice. Arrows indicate the treatment schedule.

FIGS. 16A-16C show the relative body weight change of oxaliplatin or P(G-A)-Asp-Pt in different types of cancer as treated in FIGS. 15A-15C.

FIGS. 17A and 17B show the toxicity evaluation of oxaliplatin and P(G-A)-Asp-Pt in mice. The representative gross appearance (FIG. 17A) and weight (FIG. 17B) of spleens were shown after treatment with oxaliplatin or P(G-A)-Asp-Pt.

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of ” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a polymer”, “a composition”, or “a cancer”, includes, but is not limited to, two or more such polymers, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ophthalmological disorder. The effect can be therapeutic in terms of a partial or complete cure of a disease, condition, symptom or adverse effect attributed to the disease, disorder, or condition. The term “treatment” as used herein can include any treatment of ophthalmological disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context.

“Alkyl” is a straight chain or branched saturated aliphatic hydrocarbon group. In certain embodiments, the alkyl is C₁-C_{2,} C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or -C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O-C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

The present disclosure also includes compounds with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine such as ²H, ³H, ¹¹C, ¹³C, ¹⁵N , ¹⁷O, ¹⁸O, ¹⁸F, ³¹P, ³²P, ³⁵S, ³⁶Cl, and ¹²⁵I, respectively where present. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

### Amino Acid Polymers

The present disclosure provides amino acid polymers containing sidechain groups conjugated to platinum anticancer agents. Conjugation of platinum anticancer agents to these polymers leads to increased tumor accumulation and retention, leading to improved efficacy and toxicity profiles for these agents. The amino acid polymers disclosed herein are found to enter cancer cells via endocytosis and show enhanced platinum concentrations in said cells, leading to rapid cancer cell necrosis. Further, the amino acid polymers described herein show prolonged circulation time and improved accumulation in tumor tissues, decreasing toxicity to normal tissues and improving anti-tumor efficacy.

Thus, in one aspect, the present disclosure provides an amino acid polymer comprising:
a plurality of glutamic acid residues, salts thereof, or combinations thereof;
a plurality of aspartic acid residues, salts thereof, or combinations thereof; and
a plurality of residues having the structure

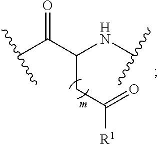

wherein:
m is 1 or 2;
R¹ is selected from

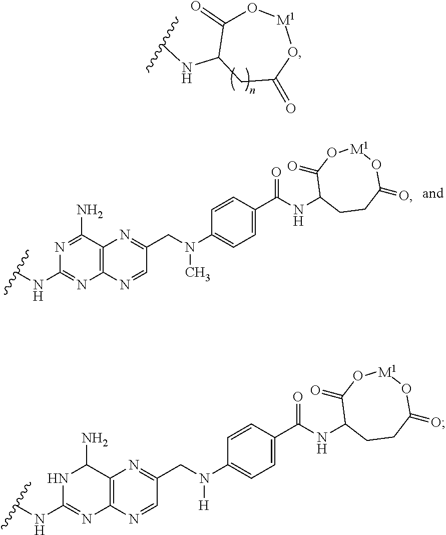

n is 1 or 2;
M¹ is Pt(NH₂R²)(NH₂R³);
R² and R³ are independently hydrogen, alkyl, or aryl; or
R² and R³ are brought together to form a cycloalkyl or heterocycle ring which may be optionally substituted with 1 or 2 alkyl groups.

In another aspect, the present disclosure provides an amino acid polymer comprising:
a plurality of glutamic acid residues, salts thereof, or combinations thereof; and
a plurality of aspartic acid residues, salts thereof, or combinations thereof;
wherein the amino acid polymer is conjugated to one or more M 2 groups having the structure:

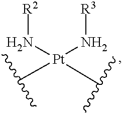

wherein each of the wavy lines represents a point of covalent attachment to the amino acid polymer, and wherein all other variables are as defined herein.

In some embodiments, the plurality of glutamic acid residues comprises L-glutamic acid residues, D-glutamic acid residues, salts thereof, or combinations thereof.

In some embodiments, the plurality of aspartic acid residues comprises L-aspartic acid residues, D-aspartic acid residues, salts thereof, or combinations thereof.

In some embodiments, the amino acid polymer has a ratio of glutamic acid residues to aspartic acid residues ranging from about 90:10 to about 10:90. In some embodiments, the ratio of glutamic acid residues to aspartic acid residues ranges from about 80:20 to 60:40. In some embodiments, the ratio of glutamic acid residues to aspartic acid residues is about 90:10, about 80:20, about 70:30, about 60:40, about 50:50, about 40:60, about 30:70, about 20:80, or about 10:90.

In some embodiments, m is 1. In some embodiments, m is 2.

In some embodiments, R¹ is

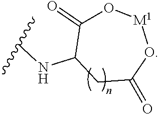

In some embodiments, n is 1. In some embodiments, n is 2.

In some embodiments, R¹ is

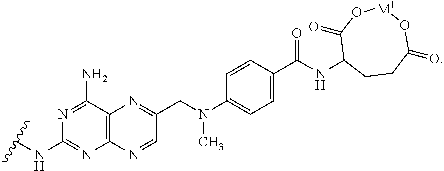

In some embodiments, R¹ is

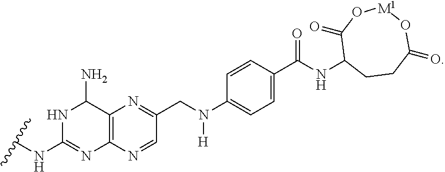

In some embodiments, R² is hydrogen. In some embodiments, R² is alkyl. In some embodiments, R² is aryl.

In some embodiments, R³ is hydrogen. In some embodiments, R³ is alkyl. In some embodiments, R³ is aryl.

In some embodiments, R² and R³ are brought together to form a cycloalkyl ring. In some embodiments, R² and R³ are brought together to form a cycloalkyl or heterocycle ring which may be optionally substituted with 1 or 2 alkyl groups

In some embodiments, M¹ is

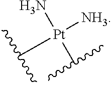

In some embodiments, M¹ is

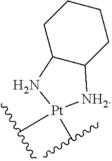

In some embodiments, M² is

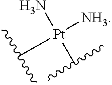

In some embodiments, M² is

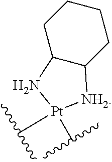

In some embodiments, the polymer has an average molecular weight ranging from about 20,000 daltons to about 30,000 daltons, for example: from about 20,000 daltons to about 29,000 daltons; from about 20,000 daltons to about 28,000 daltons; from about 20,000 daltons to about 27,000 daltons; from about 20,000 daltons to about 26,000 daltons; from about 20,000 daltons to about 25,000 daltons; from about 20,000 daltons to about 24,000 daltons; from about 20,000 daltons to about 23,000 daltons; from about 20,000 daltons to about 22,000 daltons; from about 20,000 daltons to about 21,000 daltons; from about 21,000 daltons to about 30,000 daltons; from about 21,000 daltons to about 29,000 daltons; from about 21,000 daltons to about 28,000 daltons; from about 21,000 daltons to about 27,000 daltons; from about 21,000 daltons to about 26,000 daltons; from about 21,000 daltons to about 25,000 daltons; from about 21,000 daltons to about 24,000 daltons; from about 21,000 daltons to about 23,000 daltons; from about 21,000 daltons to about 22,000 daltons; from about 22,000 daltons to about 30,000 daltons; from about 22,000 daltons to about 29,000 daltons; from about 22,000 daltons to about 28,000 daltons; from about 22,000 daltons to about 27,000 daltons; from about 22,000 daltons to about 26,000 daltons; from about 22,000 daltons to about 25,000 daltons; from about 22,000 daltons to about 24,000 daltons; from about 22,000 daltons to about 23,000 daltons; from about 23,000 daltons to about 30,000 daltons; from about 23,000 daltons to about 29,000 daltons; from about 23,000 daltons to about 28,000 daltons; from about 23,000 daltons to about 27,000 daltons; from about 23,000 daltons to about 26,000 daltons; from about 23,000 daltons to about 25,000 daltons; from about 23,000 daltons to about 24,000 daltons; from about 24,000 daltons to about 30,000 daltons; from about 24,000 daltons to about 29,000 daltons; from about 24,000 daltons to about 28,000 daltons; from about 24,000 daltons to about 27,000 daltons; from about 24,000 daltons to about 26,000 daltons; from about 24,000 daltons to about 25,000 daltons; from about 25,000 daltons to about 30,000 daltons; from about 25,000 daltons to about 29,000 daltons; from about 25,000 daltons to about 28,000 daltons; from about 25,000 daltons to about 27,000 daltons; from about 25,000 daltons to about 26,000 daltons; from about 26,000 daltons to about 30,000 daltons; from about 26,000 daltons to about 29,000 daltons; from about 26,000 daltons to about 28,000 daltons; from about 26,000 daltons to about 27,000 daltons; from about 27,000 daltons to about 30,000 daltons; from about 27,000 daltons to about 29,000 daltons; from about 27,000 daltons to about 28,000 daltons; from about 28,000 daltons to about 30,000 daltons; from about 28,000 daltons to about 29,000 daltons; and from about 29,000 daltons to about 30,000 daltons.

In some alternative embodiments, the polymer has a size ranging from about 100 nm to about 300 nm, for example the polymer may have a size ranging from about 100 nm to about 280 nm, from about 100 nm to about 260 nm, from about 100 nm to about 240 nm, from about 100 nm to about 220 nm, from about 100 nm to about 200 nm, from about 100 nm to about 180 nm, from about 100 nm to about 160 nm, from about 100 nm to about 140 nm, from about 100 nm to about 120 nm, from about 120 nm to about 300 nm, from about 120 nm to about 280 nm, from about 120 nm to about 260 nm, from about 120 nm to about 240 nm, from about 120 nm to about 220 nm, from about 120 nm to about 200 nm, from about 120 nm to about 180 nm, from about 120 nm to about 160 nm, from about 120 nm to about 140 nm, from about 140 nm to about 300 nm, from about 140 nm to about 280 nm, from about 140 nm to about 260 nm, from about 140 nm to about 240 nm, from about 140 nm to about 220 nm, from about 140 nm to about 200 nm, from about 140 nm to about 180 nm, from about 140 nm to about 160 nm, from about 160 nm to about 300 nm, from about 160 nm to about 280 nm, from about 160 nm to about 260 nm, from about 160 nm to about 240 nm, from about 160 nm to about 220 nm, from about 160 nm to about 200 nm, from about 160 nm to about 180 nm, from about 180 nm to about 300 nm, from about 180 nm to about 280 nm, from about 180 nm to about 260 nm, from about 180 nm to about 240 nm, from about 180 nm to about 220 nm, from about 180 nm to about 200 nm, from about 200 nm to about 300 nm, from about 200 nm to about 280 nm, from about 200 nm to about 260 nm, from about 200 nm to about 240 nm, from about 200 nm to about 220 nm, from about 220 nm to about 300 nm, from about 220 nm to about 280 nm, from about 220 nm to about 260 nm, from about 220 nm to about 240 nm, from about 240 nm to about 300 nm, from about 240 nm to about 280 nm, from about 240 nm to about 260 nm, from about 260 nm to about 300 nm, from about 260 nm to about 280 nm, or from about 280 nm to about 300 nm.

In some embodiments, the polymer has a size ranging from about 90 nm to about 180 nm, for example the polymer may have a size ranging from about 90 nm to about 170 nm, about 90 nm to about 160 nm, about 90 nm to about 150 nm, about 90 nm to about 140 nm, about 90 nm to about 130 nm, about 90 nm to about 120 nm, about 90 nm to about 110 nm, about 90 nm to about 100 nm, about 100 nm to about 180 nm, about 100 nm to about 170 nm, about 100 nm to about 160 nm, about 100 nm to about 150 nm, about 100 nm to about 140 nm, about 100 nm to about 130 nm, about 100 nm to about 120 nm, about 100 nm to about 110 nm, about 110 nm to about 180 nm, about 110 nm to about 170 nm, about 110 nm to about 160 nm, about 110 nm to about 150 nm, about 110 nm to about 140 nm, about 110 nm to about 130 nm, about 110 nm to about 120 nm, about 120 nm to about 180 nm, about 120 nm to about 170 nm, about 120 nm to about 160 nm, about 120 nm to about 150 nm, about 120 nm to about 140 nm, about 120 nm to about 130 nm, about 130 nm to about 180 nm, about 130 nm to about 170 nm, about 130 nm to about 160 nm, about 130 nm to about 150 nm, about 130 nm to about 140 nm, about 140 nm to about 180 nm, about 140 nm to about 170 nm, about 140 nm to about 160 nm, about 140 nm to about 150 nm, about 150 nm to about 180 nm, about 150 nm to about 170 nm, about 150 nm to about 160 nm, about 160 nm to about 180 nm, about 160 nm to about 170 nm, and about 170 nm to about 180 nm.

In some embodiments, the polymer has a zeta potential ranging from about —30 mV to about —55 mV, for example of about —30 mV, about —32 my, about —34 mV, about —35 mV, about —36 mV, about —38 mV, about —40 mV, about —42 mV, about —44 mV, about —45 mV, about —46 mV, about —48 mV, about —50 mV, about —52 mV, about —54 mV, or about —55 mV.

### Methods of Treatment

The present disclose also provides methods for treating or preventing a cancer in a subject, the methods comprising administering to the subject a therapeutically effective amount of a compound or pharmaceutical composition described herein. The methods can further comprise administering a second compound or composition, such as, for example, anticancer agents or anti-inflammatory agents. Additionally, the method may further comprise administering an effective amount of ionizing radiation to the subject.

Methods of killing a tumor cell are also provided comprising contacting a tumor cell with an effective amount of a compound or pharmaceutical composition as described herein.

In some embodiments, the subject to be treated may have been previously administering a platinum anticancer agent. In other embodiments, the cancer to be treated may have developed resistance to a platinum anticancer agents. Representative examples of platinum anticancer agents include, but are not limited to, cisplatin, carboplatin, oxaliplatin, and nedaplatin.

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma(MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenstrom macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndromes, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDs-related cancers such as Kaposi sarcoma; epithelioid hemangioendothelioma; desmoplastic small round cell tumor; and liposarcoma.

### Methods of Administration

The compounds as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and an excipient of some sort may be useful in a variety of medical and non-medical applications. For example, pharmaceutical compositions comprising an active compound and an excipient may be useful for the treatment or prevention of an infection with a Mycobacterium.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some embodiments, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen- free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxy ethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl -sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, 0 absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

For the treatment of oncological disorders, compounds, agents, and compositions described herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as Adriamycin or bleomycin, topoisomerase inhibitors such as etoposide or camptothecin, antiangiogenic agents such as angiostatin, antiestrogens such as tamoxifen, and/or other anticancer drugs or antibodies, such as, for example, imatinib or trastuzumab. These other substances or radiation treatments can be given at the same time as or at different times from the compounds disclosed herein. Examples of other suitable chemotherapeutic agents include, but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine, topotexan, treosulfan, vinblastine, vincristine, vindesine, or vinorelbine. Examples of suitable immunotherapeutic agents include, but are not limited to alemtuzumab, cetucimab, gemtuzumab, iodine 131 tositumomab, rituximab, or trastuzumab. Cytotoxic agents include, for example, radioactive isotopes or toxins of bacterial, fungal, plant, or animal origin. Also disclosed are methods for treating an oncological disorder comprising administering an effective amount of a compound or compositions disclosed herein prior to, subsequent to, and/or in combination with administration of a chemotherapeutic agent, an immunotherapeutic agent, a radiotherapeutic agent, or radiotherapy. In some embodiments, compounds, agents, and compositions described herein can be administered to a patient in need of treatment prior to, subsequent to, or in combination with an immune checkpoint inhibitor. The immune checkpoint inhibitor may comprise a PD-1 inhibitor, a PD-L₁ inhibitor, or a CTLA-4 inhibitor. Representative examples of immune checkpoint inhibitors which may be used include, but are not limited to, ipilimumab, nivolumab, pembrolizumab, atezolizumab, avelumab, durvalumab, and cemiplimab.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described processes. Only reasonable and routine experimentation will be required to optimize such process conditions.

### Synthesis and Characterization of Amino Acid Polymer/Platinum Conjugates

The following amino acid polymer/platinum conjugates were prepared:

| Abbreviation | Full Name |
|---|---|
| P(G-A)-Asp-Pt | Poly L-Glutamic and poly L-Aspartic acid |
| | (Glu:Asp = 7:3)-L-Aspartic-Platinum |
| P(G-A)-Pt | Poly L-Glutamic and poly L-Aspartic acid |
| | (Glu:Asp = 7:3)-Platinum |
| PAA-Asp-Pt | Poly L-Aspartic acid-L-Aspartic-Platinum |
| PGA-Asp-Pt | Poly-L-Glutamic acid-L-Aspartic-Platinum |
| Asp-Pt | L-Aspartic-Platinum |
| P(G-A)-Glu-Pt | Poly L-Glutamic and poly L-Aspartic acid |
| | (Glu:Asp = 7:3)-L-Glutamic-Platinum |
| P(G-A)-D-Asp-Pt | Poly L-Glutamic and poly L-Aspartic acid |
| | (Glu:Asp = 7:3)-D-Aspartic-Platinum |

P(G-A)-Asp-Pt was prepared according to the following scheme:

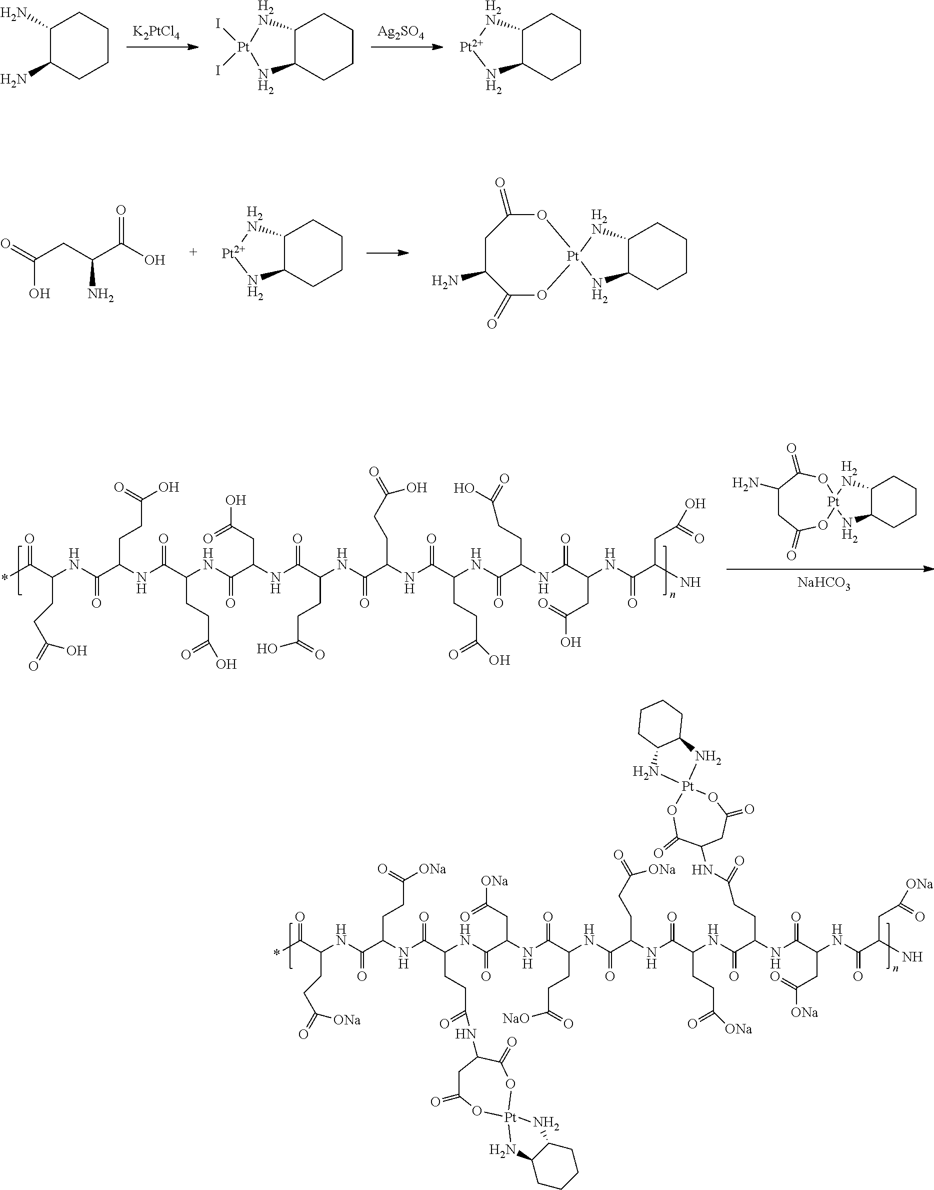

P(G-A)-Pt was prepared according to the following scheme:

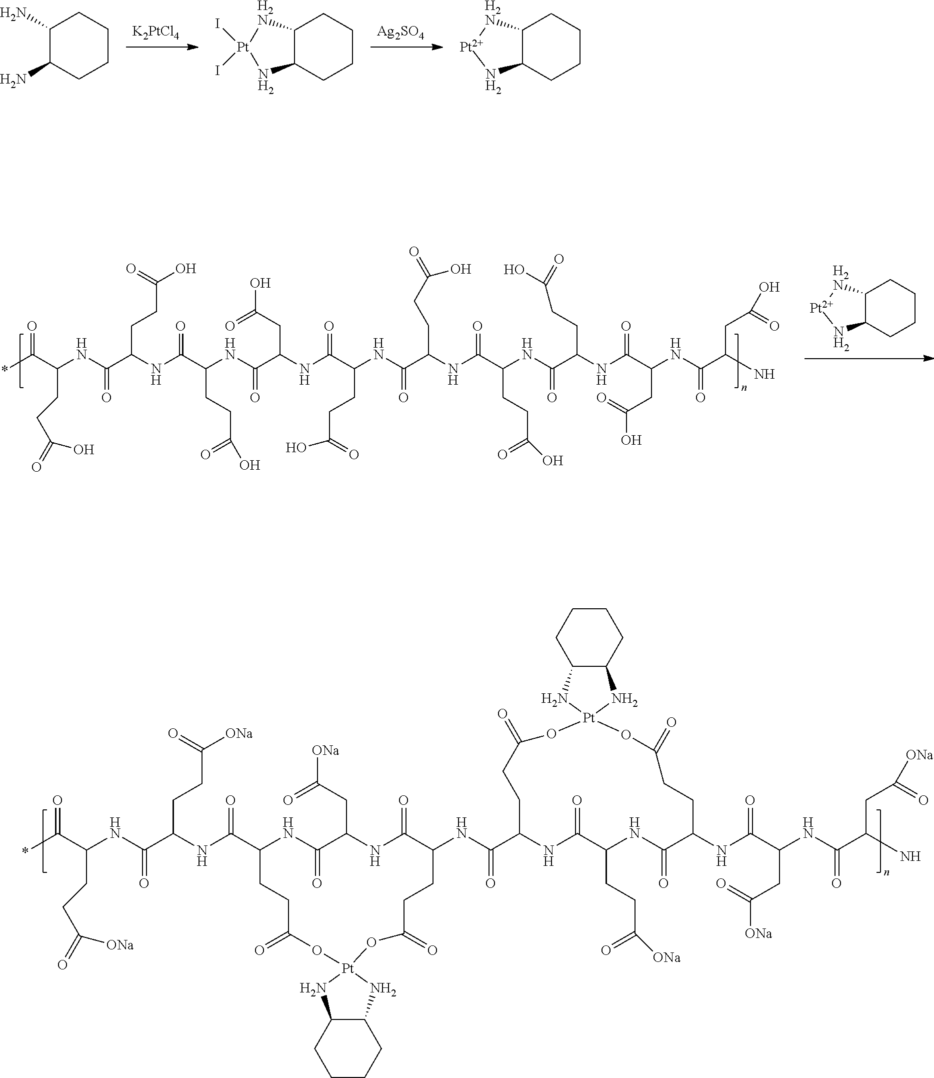

The following characterization data were obtained for the amino acid polymer/platinum conjugates prepared:

| | Carrier molecular | Size | Zeta |
|---|---|---|---|
| Conjugate | weight (D) | (nm) | potential (mV) |
| PGA-Asp-Pt | 26,000 | 100-300 | −36 |
| P(G-A)-Asp-Pt | 25,200 | 100-300 | −50 |
| P(G-A)-Pt | 25,200 | 100-300 | −48 |
| PAA-Asp-Pt | 27,000 | 100-300 | −45 |

As can be seen from the above data, the different platinum derivatives synthesized have different sizes and zeta potentials. The size of the platinum derivatives ranges from around 100 to 300 nm, and all of the derivatives carry a negative charge.

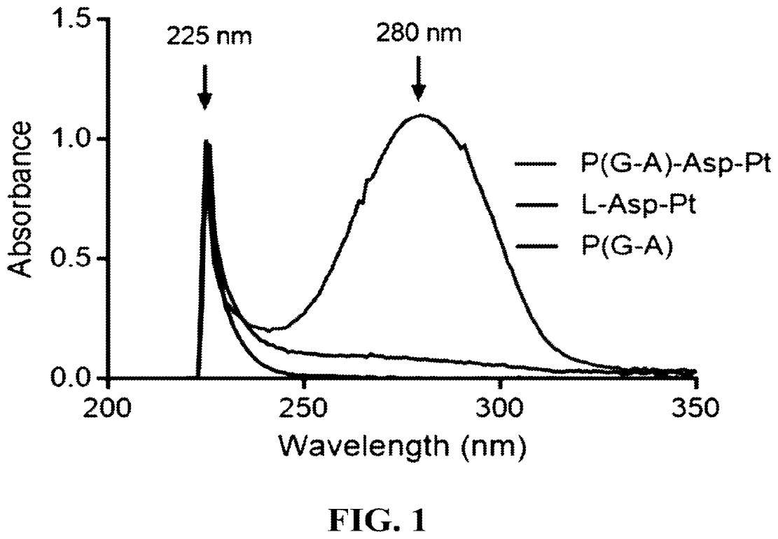

UV-visible spectra in water were obtained for P(G-A), Asp-Pt, and P(G-A)-Asp-Pt (FIG. 1). The λₘₐₓ for P(G-A)-COONa, Asp-Pt, and P(G-A)-Asp-Pt was found to be 225 nm, 225 nm, and 280 nm, respectively. An increase in λₘₐₓ of P(G-A)-Asp-Pt indicates that P(G-A) and Asp-Pt are conjugated. Further, the UV-visible spectrum for P(G-A)-Asp-Pt shows that there is no free Asp-Pt found in that solution. The absorption peak for P(G-A)-Asp-Pt may also be used for quantification of this compound.

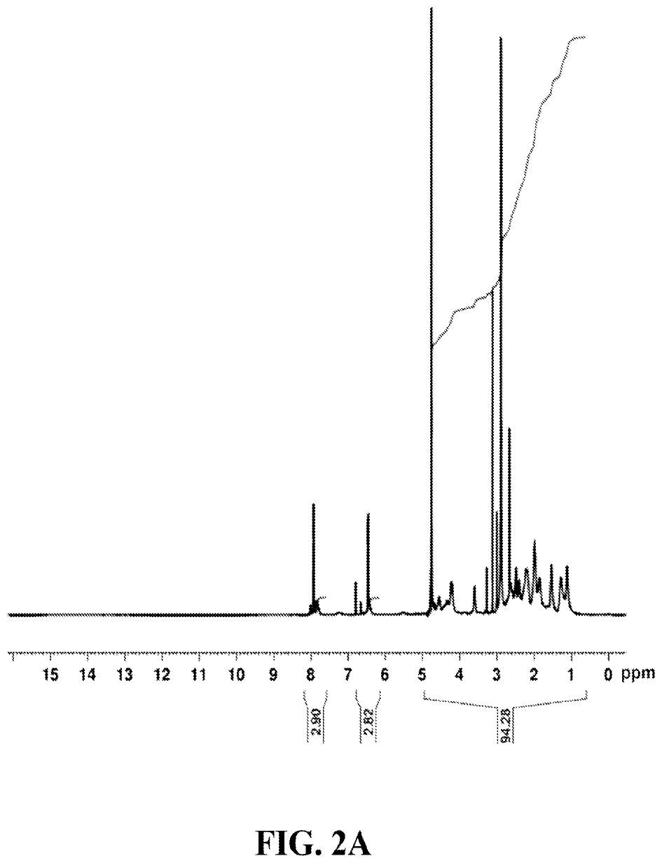

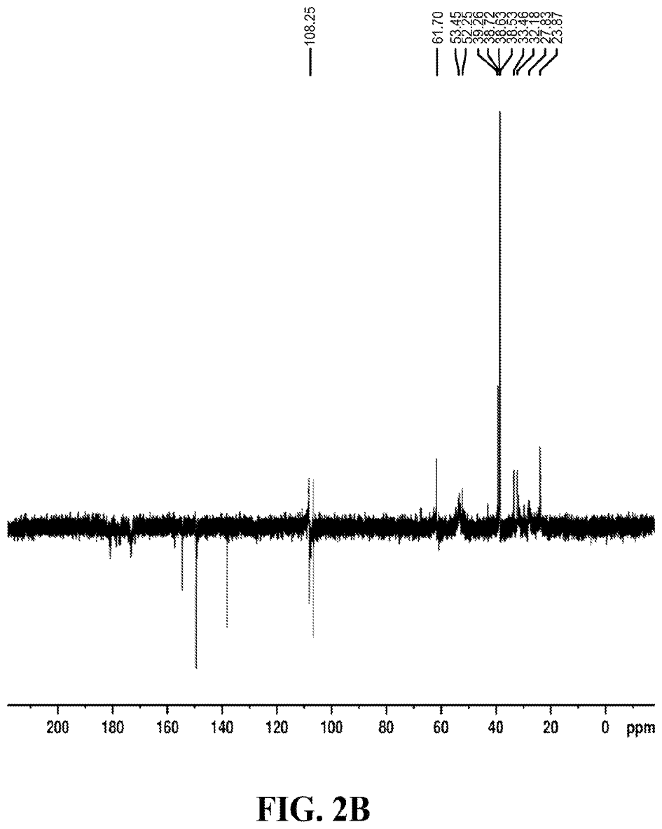

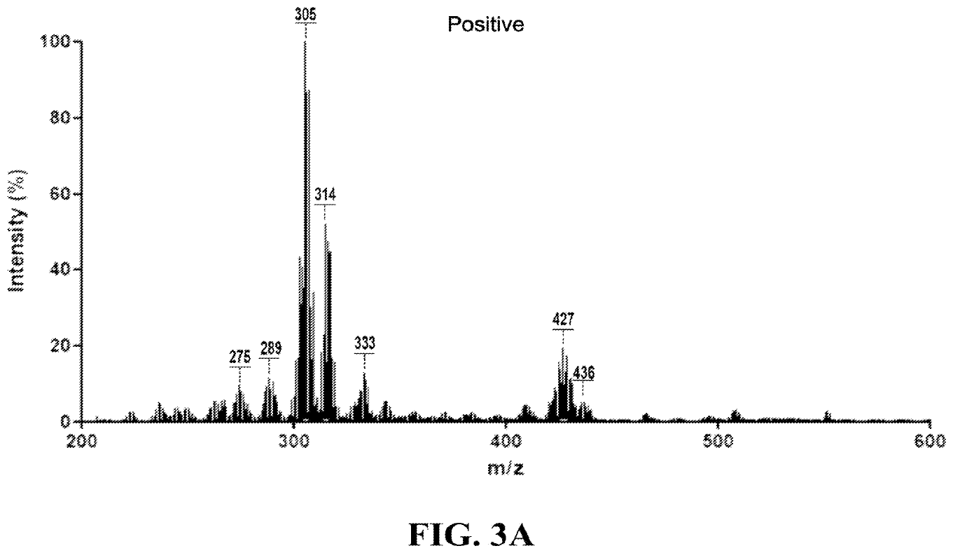

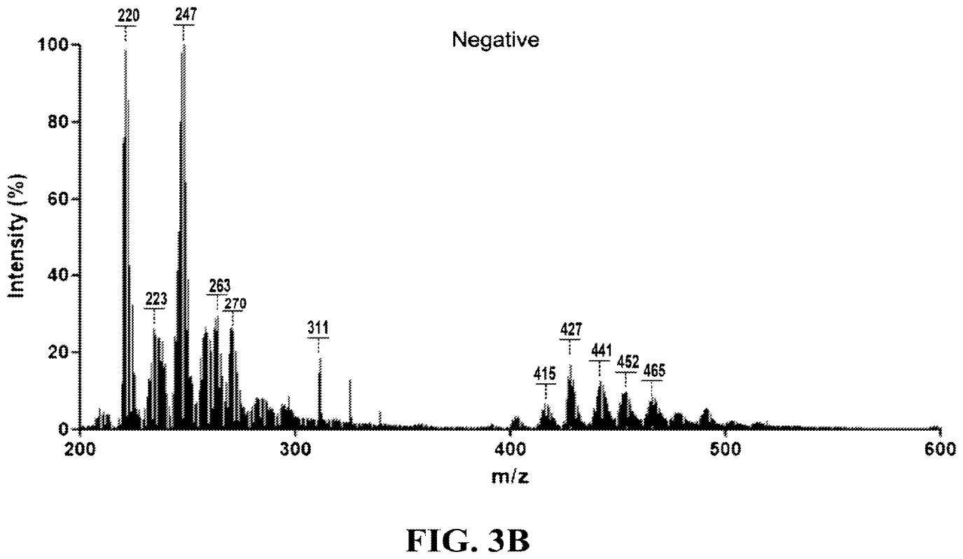

¹H-NMR, ¹³C-NMR spectra and mass spectrograms were also obtained for P(G-A)-Asp-Pt (FIGS. 2A and 2B, FIGS. 3A and 3B), which further confirmed successful conjugation.

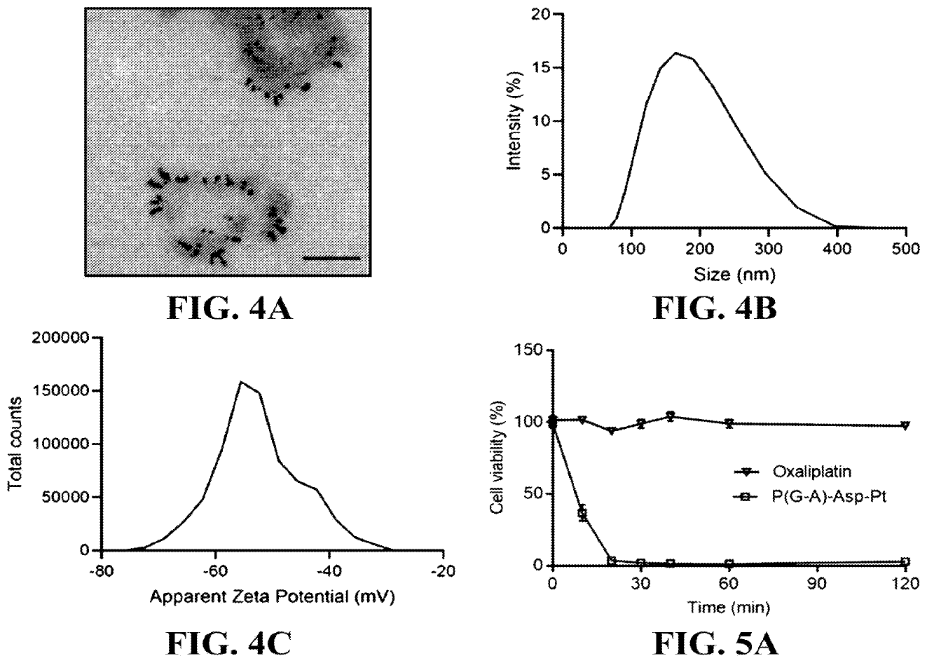

The size of P(G-A)-Asp-Pt was around 100-300 nm and the Zeta potential was around −50 mV (FIGS. 4A-4C).

### P(G-A)-Asp-Pt Quickly Trigger Cancer Cell Death In Vitro

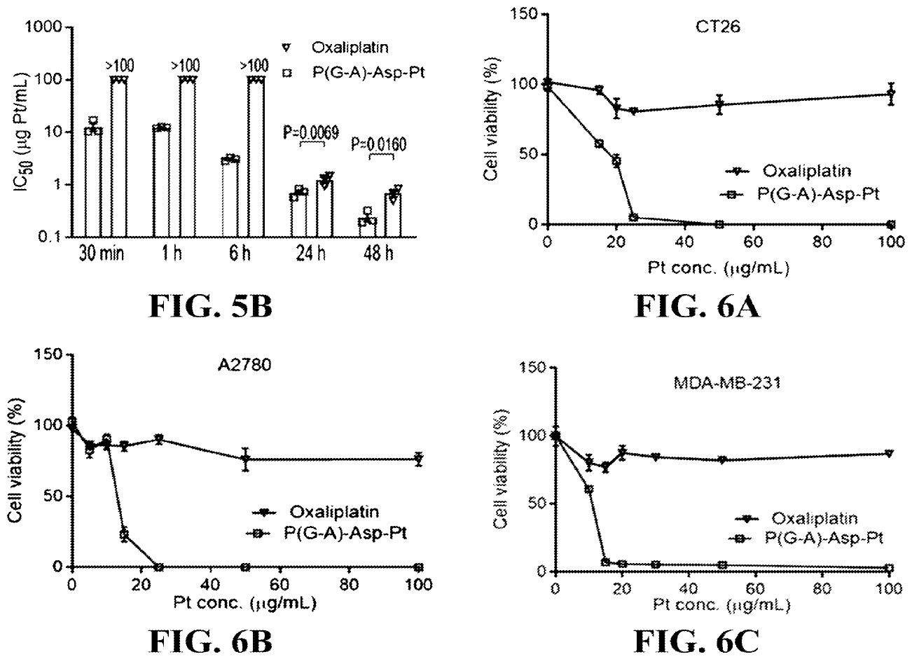

5×10³ CT26 cells/well were seeded in a 96-well plate and cultured overnight. The CT26 cells were treated with oxaliplatin or P(G-A)-Asp-Pt in different concentration of Pt for different time points. Cell viability was detected by CCK8 assay. The concentration of each of the platinum derivatives was calculated based on platinum concentration. The results of this study are found in FIGS. 5A and 5B. P(G-A)-Asp-Pt was found to induce rapid cell death in as short as 30 min with a time-dependent manner, while oxaliplatin had little effect within that time interval (FIG. 5A). P(G-A)-Asp-Pt showed better cell killing efficacy on CT26 cells compared with oxaliplatin with a lower IC₅₀ in 24 and 48 h (FIG. 5B).

### P(G-A)-Asp-Pt has a Broad Anti-Tumor Spectrum as it's Effective on Multiple Types of Cancer Cells

CT26, A2780 or MDA-MB-231 cells were seeded in a 96-well plate and cultured overnight. These cells were treated with oxaliplatin or P(G-A)-Asp-Pt in different concentrations of Pt for 30 min. P(G-A)-Asp-Pt was able to kill these cancer cells (FIG. 6A-6C) in 30 min while oxaliplatin had little effect, demonstrating the potent efficacy and broad anti-tumor spectrum of P(G-A)-Asp-Pt.

### Cytotoxicity of Amino Acid Polymer/Platinum Conjugates to CT26 Cells

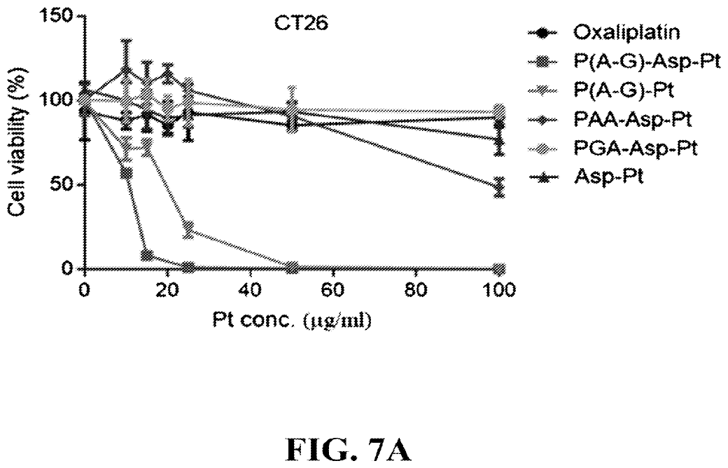

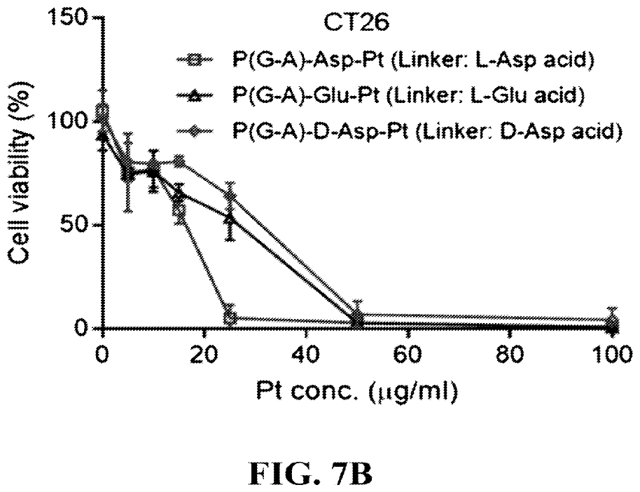

5×10³ CT26 cells/well were seeded in a 96-well plate and cultured overnight, followed by treatment with oxaliplatin, P(G-A)-Asp-Pt, P(G-A)-Pt, PAA-Asp-Pt, PGA-Asp-Pt, and Asp-Pt, respectively, for 30 min. The concentration of each of the platinum derivatives was calculated based on platinum concentration. The results of this study are found in FIG. 7A. P(G-A)-Asp-Pt and P(G-A)-Pt showed the best cytotoxicity to CT26 cells compared to the other platinum derivatives tested. Further, a glutamic acid to aspartic acid ratio of 7:3 in the backbone had the best efficacy to inhibit cell growth compared to derivatives containing only glutamic acid or aspartic acid in the backbone.

P(G-A)-Asp-Pt shows better efficacy that P(G-A)-Pt (FIG. 7A), demonstrating the importance of L-Asp acid as a linker. L-Asp acid as a linker was further found to be more efficient at killing cancer cells than L-glu acid or D-Asp acid, as P(G-A)-Asp-Pt shows better efficacy that P(G-A)-Glu-Pt and P(G-A)-D-Asp-Pt. The results of this study are found in FIG. 7B.

### P(G-A)-Asp-Pt Overcomes Drug Resistance In Vitro

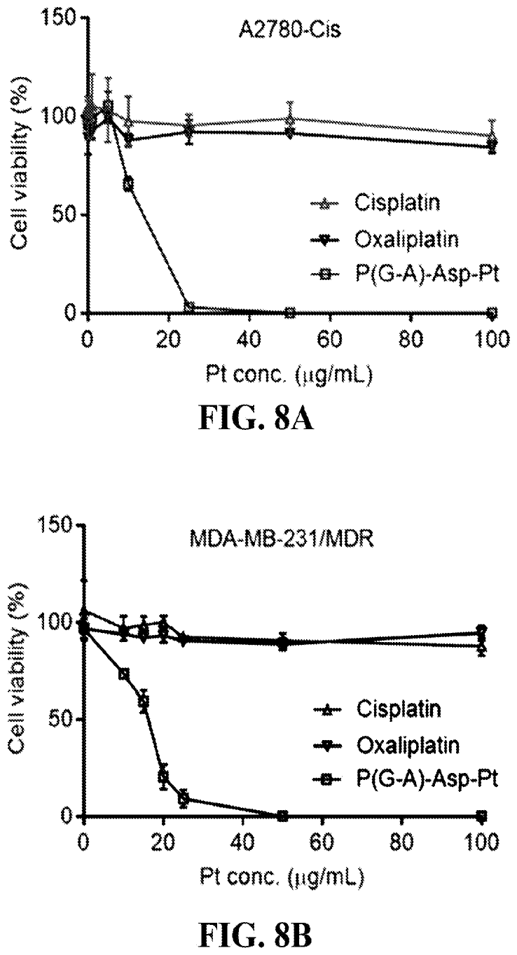

A2780 cisplatin-resistant cells (A2780-Cis) and multiple drug resistant MDA-MB-231 cells (MDA-MB-231/MDR) were employed as models of drug resistant cells. 5×10³ A2780-Cis or MDA-MB-231/MDR cells/well were seeded in a 96-well plate and cultured overnight. These cells were treated with oxaliplatin or P(G-A)-Asp-Pt at different concentrations of Pt for 30 min. The results of this study are found in FIGS. 8A and 8B. P(G-A)-Asp-Pt was able to kill these drug resistant cells in 30 min while both oxaliplatin and cisplatin had little effect.

### P(G-A)-Asp-Pt is More Sensitive to Cancer Cells Compared With Normal Cells

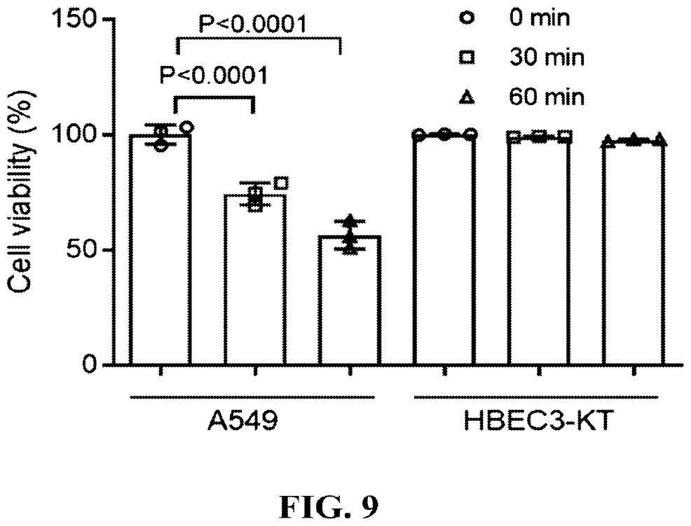

A549 cancer cells or HBEC-KT normal cells were treated with P(G-A)-Asp-Pt for 30-60 min, the results of which may be found in FIG. 9. P(G-A)-Asp-Pt selectively kills A549 cancer cells in vitro but has little effect on normal HBEC3-KT cells. These results indicate that P(G-A)-Asp-Pt is more sensitive to cancer cells compared with normal cells.

### P(G-A)-Asp-Pt Efficiently Enters Cells via Endocytosis

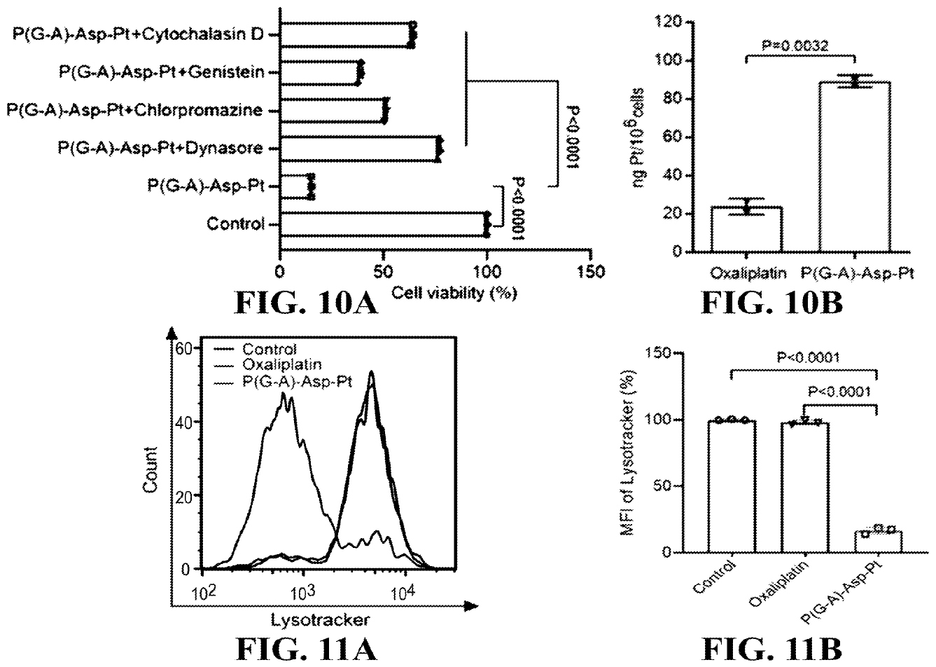

CT26 cells were pretreated with inhibitors for endocytosis/micropinocytosis (Dynasore, chlorpromazine, genistein and cytochalasin D) for 40 min before incubation with P(G-A)-Asp-Pt at 25 μg (Pt)/mL for 30 min. The results are shown in FIG. 10A. The administration of these inhibitors can partially restore cell viability after P(G-A)-Asp-Pt treatment, demonstrating that P(G-A)-Asp-Pt could enter the cell via endocytosis.

Pt concentration in CT26 cells was analyzed after treatment with oxaliplatin or P(G-A)-Asp-Pt after incubation with oxaliplatin or P(G-A)-Asp-Pt at 10 μg (Pt)/mL for 1 h. P(G-A)-Asp-Pt showed enhanced Pt accumulation inside cancer cells compared with oxaliplatin (FIG. 10B), indicating increased cellular uptake of P(G-A)-Asp-Pt.

### P(G-A)-Asp-Pt Induced Lysosomal Rupture

CT26 cells were seeded in a 6-well plate and cultured overnight. Cells were treated with oxaliplatin or P(G-A)-Asp-Pt for 1 h. Cells were then stained with LysoTracker deep red (100 nM) for 40 min. Cells were analyzed by flow cytometry. P(G-A)-Asp-Pt treatment reduced LysoTracker signal, indicating lysosomal rupture, while oxaliplatin had little effect. The results are provided in FIGS. 11A and 11B.

### P(G-A)-Asp-Pt Induced Cancer Cell Necrosis

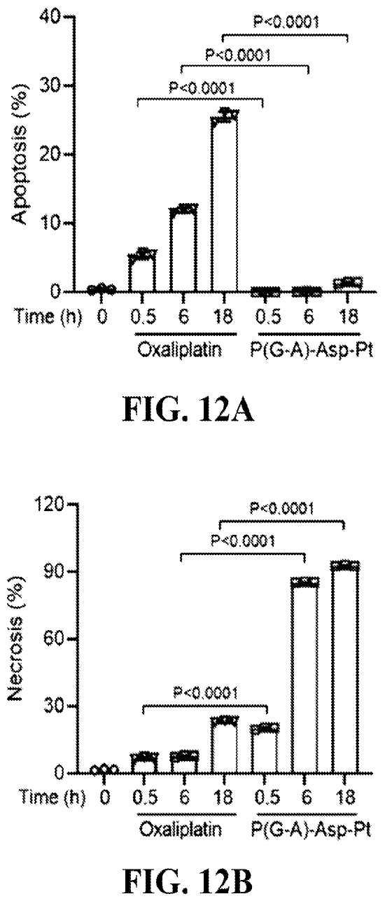

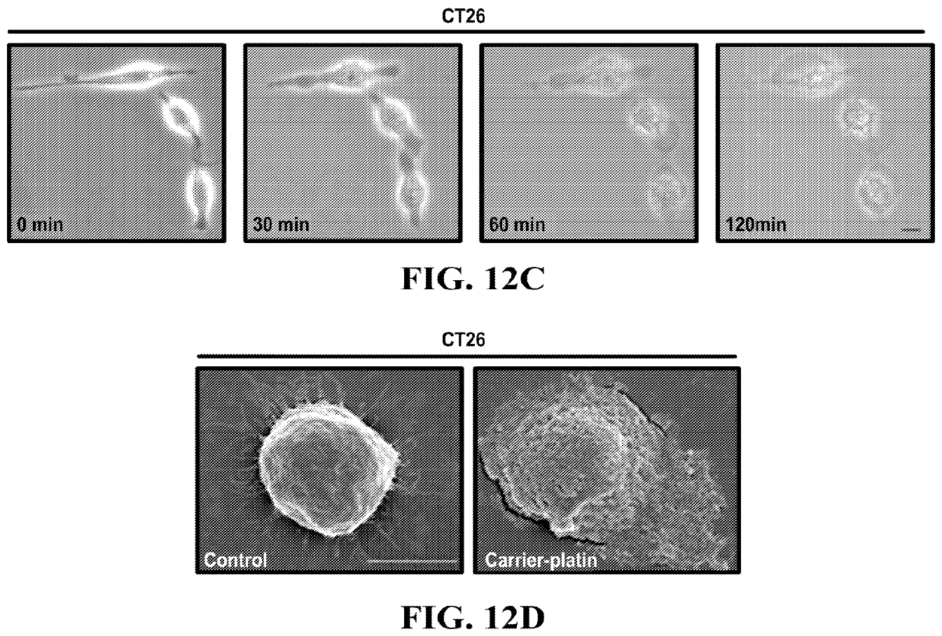

CT26 cells were seeded in a 6-well plate and cultured overnight. Flow cytometry analysis of the Annexin-V/propidium iodide (PI) staining of CT26 cells after treatment with oxaliplatin or P(G-A)-Asp-Pt at 15 μg (Pt)/mL at different times. Apoptosis: Annexin V⁺ PI⁻, Necrosis: PI⁺. The results are provided in FIGS. 12A and 12B. P(G-A)-Asp-Pt triggered cancer cell necrosis quickly in 30 min, while oxaliplatin induced cell apoptosis gradually from 0.5 h to 18 h.

Obvious cancer cell swelling and disrupted plasma membrane of CT26 cells was observed after short-time treatment with P(G-A)-Asp-Pt (FIGS. 12C and 12D), which is a typical change during cell necrosis. These results indicate that P(G-A)-Asp-Pt induced cancer cell necrosis while oxaliplatin induce apoptosis.

### P(G-A)-Asp-Pt Shows Prolonged Circulation Time

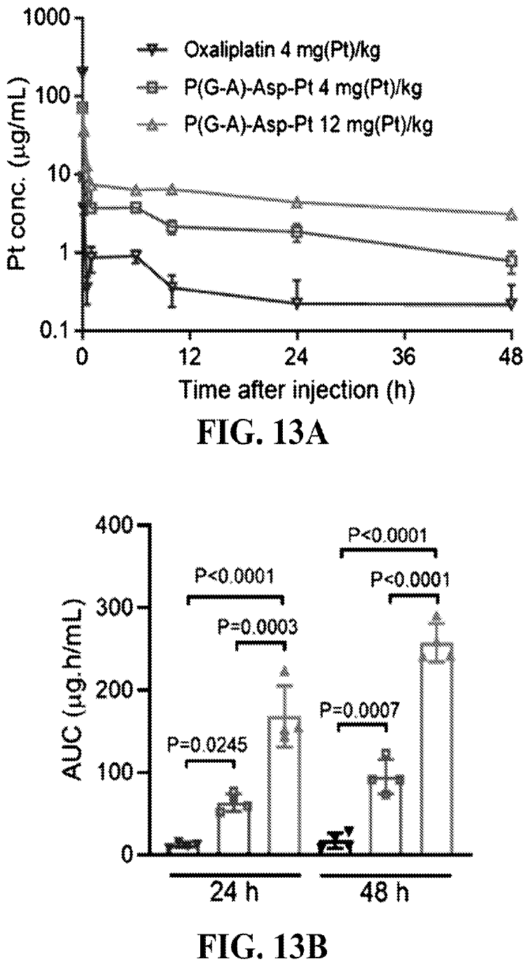

Tumor-free mice was treated with oxaliplatin or P(G-A)-Asp-Pt. The dosage was calculated based on Pt. Plasma samples were collected at different time points. Pt content was measured by ICP-OES. The results are shown in FIGS. 13A and 13B. Oxaliplatin was quickly cleared from plasma. In comparison, mice treated with 4 mg (Pt)/kg Pt-equivalent dosage of P(G-A)-Asp-Pt showed significantly prolonged circulation time and a higher AUC. In addition, P(G-A)-Asp-Pt also exhibited a dose-AUC relationship as the high dose P(G-A)-Asp-Pt treatment group had a higher AUC than the low dose group. These results demonstrated prolonged circulation time with P(G-A)-Asp-Pt versus oxaliplatin.

### P(G-A)-Asp-Pt Shows Improved Accumulation in Tumor Tissues

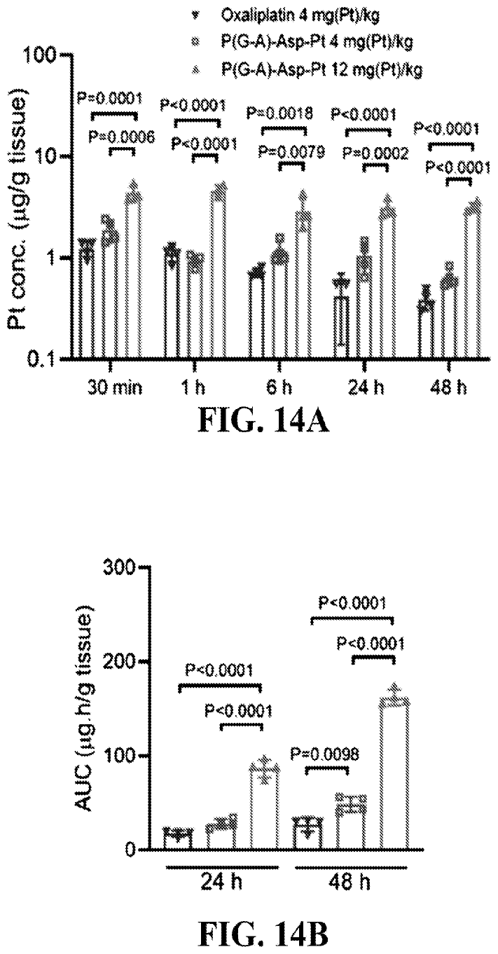

CT26-tumor bearing mice were intravenously injected with oxaliplatin or P(G-A)-Asp-Pt. The dosage was calculated based on Pt. Tumor samples were harvested at different time points for the detection of platinum concentration inside the tumor via ICP-OES, the results of which are shown in FIGS. 14A and 14B. Pt concentration in tumors rapidly dropped over time after oxaliplatin treatment, while it was significantly higher when treating with same dosage of P(G-A)-Asp-Pt. The 12 mg (Pt)/kg P(G-A)-Asp-Pt group showed dramatic tumor accumulation with increased concentration. P(G-A)-Asp-Pt achieved Pt concentrations in tumor well above those in cell culture IC₅₀ at 24 or 48 h (FIG. 5B) in P(G-A)-Asp-Pt treatment groups. These results indicate that P(G-A)-Asp-Pt shows improved accumulation in tumor tissues

### P(G-A)-Asp-Pt Inhibited Multiple Types of Tumor Growth In Vivo

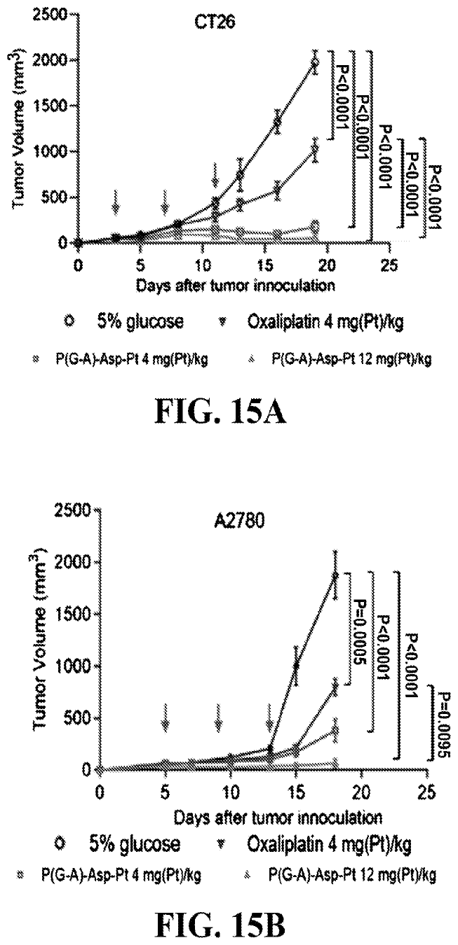

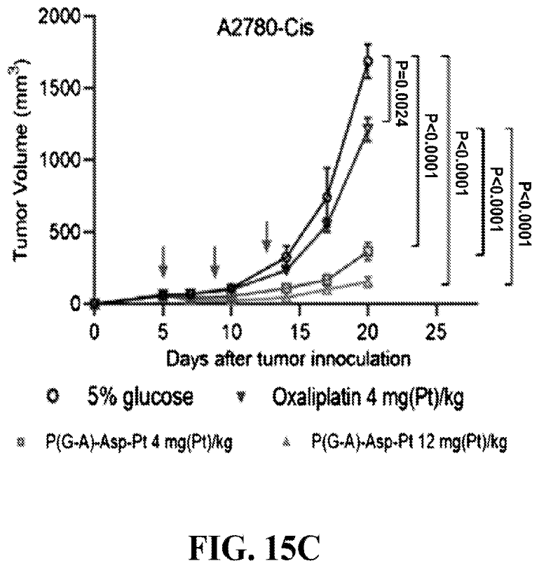

CT26, A2780 or A2780-Cis tumor-bearing mice were intravenously injected with 5% glucose, oxaliplatin and P(G-A)-Asp-Pt. The dosage was calculated based on Pt. Results are shown in FIGS. 15A-15C. Oxaliplatin partially slowed CT26 and A2780 tumor growth compared to the control group (5% glucose), but the tumors kept growing (FIGS. 15A and 15B). P(G-A)-Asp-Pt significantly inhibited tumor growth compared to the same oxaliplatin dosage. High dose P(G-A)-Asp-Pt treatment completely inhibited the growth of both CT26 and A2780 tumors with over 40% mice had complete response. For the drug resistant tumors, although oxaliplatin still showed partial anti-tumor efficacy on A2780-Cis tumors compared with the control group (FIG. 15C), it was dramatically less efficient than in parental A2780 tumors (FIG. 15B). However, P(G-A)-Asp-Pt still significantly prohibited A2780-Cis tumor growth (FIG. 15C). Collectively, these results show that P(G-A)-Asp-Pt exhibits superior anti-tumor efficacy for multiple cancer types and can conquer drug resistance in vivo.

### P(G-A)-Asp-Pt Shows Satisfactory Toxicity Profile In Vivo

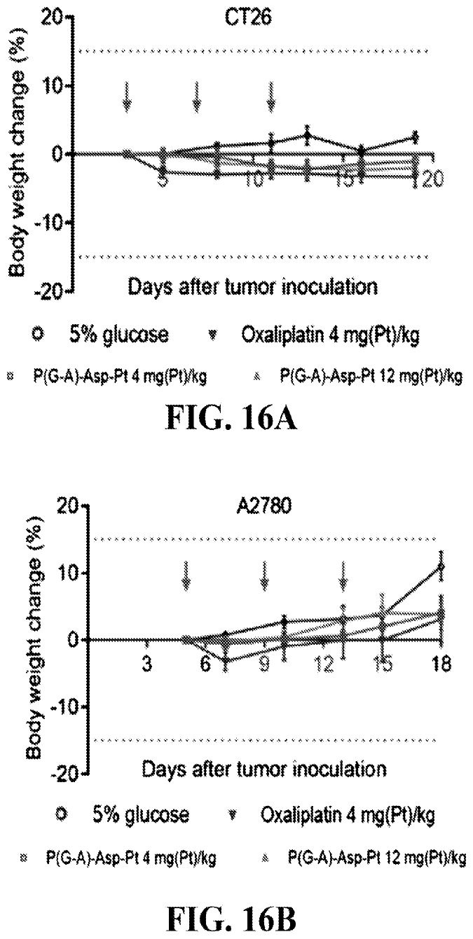

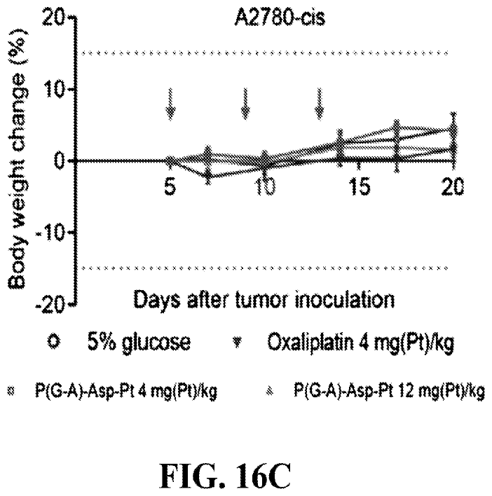

Body weight decreased significantly (more than 15%) in mice following a single 8 mg (Pt)/kg oxaliplatin injection, indicating the low tolerance for oxaliplatin. Generally, no body weight loss was observed in any of the treatment groups during the efficacy study (FIG. 16A-16C). Mice receiving high dose P(G-A)-Asp-Pt (3-fold higher than the oxaliplatin dose) showed stable bodyweight, indicating the improved tolerance of P(G-A)-Asp-Pt in vivo.

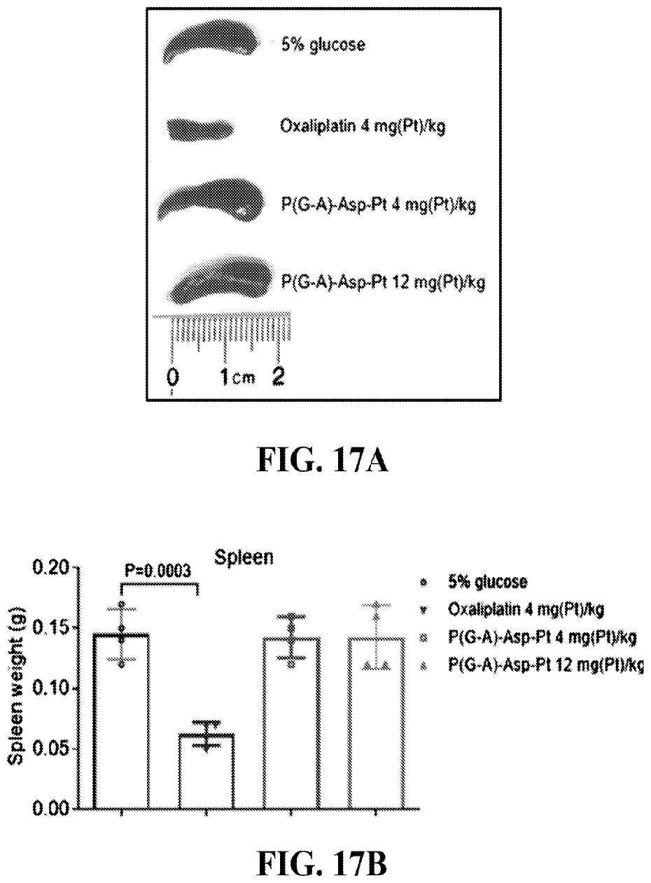

Moreover, spleens in the oxaliplatin group were dramatically shrunken with decreased weight (FIG. 17A-17B), indicating severe spleen toxicity. However, P(G-A)-Asp-Pt showed minimal changes in spleen. The above results demonstrate that P(G-A)-Asp-Pt shows satisfactory toxicity profile in vivo and is safe to use in tumor treatment.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. An amino acid polymer comprising:
a plurality of glutamic acid residues, salts thereof, or combinations thereof;
a plurality of aspartic acid residues, salts thereof, or combinations thereof; and
a plurality of residues having the structure 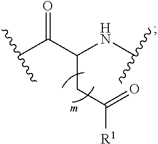
wherein:
m is 1 or 2;
R¹ is selected from 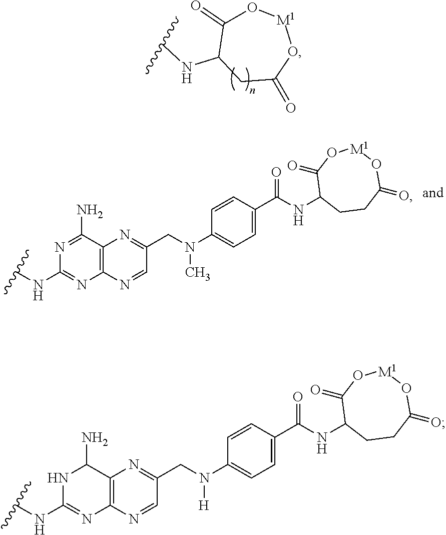
n is 1 or 2;
M¹ is Pt(NH₂R²)(NH₂R³);
R² and R³ are independently hydrogen, alkyl, or aryl; or
R² and R² are brought together to form a cycloalkyl or heterocycle ring which may be optionally substituted with 1 or 2 alkyl groups.

2. The amino acid polymer of claim 1, wherein the plurality of glutamic acid residues comprises L-glutamic acid residues, D-glutamic acid residues, salts thereof, or combinations thereof; and
wherein the plurality of aspartic acid residues comprises D-aspartic acid residues, L-aspartic acid residues, salts thereof, or combinations thereof.

3. (canceled)

4. The amino acid polymer of claim 1 having a ratio of glutamic acid residues to aspartic acid residues ranging from about 9:1 to about 1:9.

5-6. (canceled)

7. The amino acid polymer of claim 1, wherein m is 1.

8. The amino acid polymer of claim 1, wherein m is 2.

9. The amino acid polymer of claim 1, wherein R¹ is

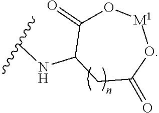

10. The amino acid polymer of claim 9, wherein n is 1.

11. The amino acid polymer of claim 9, wherein n is 2.

12. The amino acid polymer of claim 1, wherein R¹ is

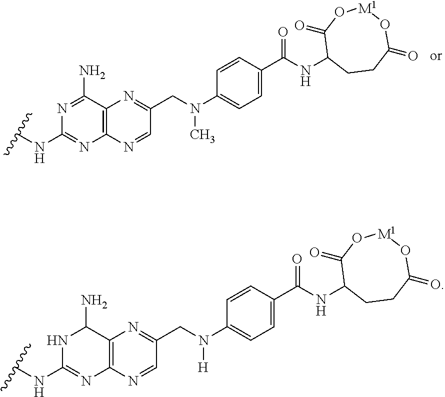

13. (canceled)

14. The amino acid polymer of claim 1, wherein M¹ is

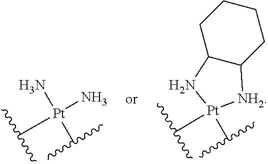

15. (canceled)

16. An amino acid polymer comprising:
a plurality of glutamic acid residues, salts thereof, or combinations thereof; and
a plurality of aspartic acid residues, salts thereof, or combinations thereof;
wherein the amino acid polymer is conjugated to one or more M² groups having the structure: 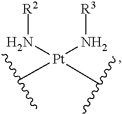
wherein each of the wavy lines represents a point of covalent attachment to the amino acid polymer, and
wherein R² and R³ are independently hydrogen, alkyl, or aryl; or
R² and R² are brought together to form a cycloalkyl or heterocycle ring which may be optionally substituted with 1 or 2 alkyl groups.

17-23. (canceled)

24. The amino acid polymer of claim 1, wherein the polymer has an average molecular weight ranging from about 20,000 daltons to about 30,000 daltons.

25. The amino acid polymer of claim 1, wherein the polymer has a size ranging from about 100 nm to about 300 nm.

26. The amino acid polymer of claim 1, wherein the polymer has a zeta potential ranging from about −30 mV to about −55 mV.

27. A pharmaceutical composition comprising an amino acid polymer of claim 1 and a pharmaceutically acceptable carrier.

28. A method of treating a cancer in a subject in need thereof comprising administering a therapeutically effective amount of an amino acid polymer of claim 1.

29. The method of claim 28, wherein the amino acid polymer is administered in combination or alternation with one or more additional anticancer agents.

30. (canceled)

31. The method of claim 28, wherein the amino acid polymer is administered in combination or alternation with radiotherapy.

32. A method of treating a cancer in a subject in need thereof, wherein the subject has been previously administered a platinum anticancer agent, the method comprising administering a therapeutically amount of an amino acid polymer of claim 1.

33. (canceled)

34. A method of treating a cancer in subject in need thereof, wherein the cancer has developed resistance to a platinum anticancer agent, the method comprising administering a therapeutically effective amount of an amino acid polymer of claim 1.

35-36. (canceled)
